(19) United States
(12) Patent Application Publication
(10) Pub. No.: US 2011/0015717 A1
(43) Pub. Date: Jan. 20, 2011

(54) CONSTRUCTIVE DISPOSITION INTRODUCED IN INDEFLATOR DEVICE APPLIED TO ANGIOPLASTY PROCEDURES IN THE BI-ARTERIAL TREATMENT AND THE KISSING-BALLOON TECHNIQUE

(75) Inventor: Fábio Sandole de Brito, JR., São Paulo (BR)

Correspondence Address: The Law Office of Michael E. Kondoudis, 888 16th Street, N.W., Suite 800, Washington, DC, 20006, US

(73) Assignee: Melchiades da Cunha Neto, Goiania (BR)

(21) Appl. No.: 12/838,519

(22) Filed: Jul. 19, 2010

(30) Foreign Application Priority Data
Jul. 20, 2009 (BR) MU 8902041-3

(51) Int. Cl. A61F 2/84; A61M 25/10

(52) U.S. Cl. 623/1.11; 604/98.01

(57) ABSTRACT

Embodiments of the present invention relate to constructive disposition introduced in an indeflator device applied in coronary angioplasty interventions for bi-arterial treatment and the kissing balloon technique. New introduced disposition in the indeflator and/or insuflator device is used in coronary angioplasty interventions especially to treat multiple bifurcation and bi-arterial lesions at the same time. Administering liquids or fluids at the indicated pressure used during surgical interventions, to inflate, control the pressure and also deflate balloon catheters, where this procedure is widely used in angioplasty interventions, enables the implementation of double endoprosthetic, also known as stent.

### BACKGROUND

1. Technical Field

Embodiments of the present invention relate to angioplasty and, more particularly, to a new introduced disposition in an indeflator, and/or standard inflation device, used in coronary angioplasty interventions specially to treat bifurcation lesions and/or multiple lesions. Administering liquids or fluids in the prescribed pressure used during surgical procedures, to inflate, control pressure and also deflate balloon catheters, where this procedure is widely used in angiographic procedures, enabling the placement of a double endoprosthetic, also known as stent.

2. Description of Related Art

The present patent request of the utility epigraph model in the title, regards an incremental evolutionary solution in indeflator devices, also known as standard inflating device, widely used in surgical procedures, specially in coronary angioplasty interventions, vascular, radiology or neurological where the mentioned device receives upgrade of a constructive nature to improve the kissing-balloon technique in the treatment for double coronary lesion allowing the inflation or administration of liquids and/or fluids simultaneously to two catheters of the “balloon” type.

The introduced constructive concept also bestows upon the device greater reliability, in terms of greater pressure control in the balloons, fact that enables better fluid and or liquid administration. Furthermore, lower cost to the user, since this technique up until used two inflators, now the user (doctor) can minimize the procedure elapsed time, and even perform an angioplasty procedure without an assistant, besides having greater awareness in which of the arteries the balloons are.

It can be concluded that the improvement made in the indeflator device, here claimed, is comprised of inventive activity with commercial and industrial applications, meeting the patent requirements, specially, as a utility model, according to the article in the Law that is in effect.

Coronary disease can be defined by the presence of important obstructive lesions in at least two of the three coronary location of myocardial irrigation: right coronary arteries, anterior descending and circumflex. However, there is no uniformity among studies regarding the degree of obstruction of the luminal diameter superior to 50% limits the reserve of coronary flow, valued in most studies. Other studies value only the obstructions superior to 70%, those are usually the ones the cause clinically important ischemia and that require revascularization.

### Technique Fundamentals

The treatment of bifurcation lesions comprise in a subgroup of patients, clinical and anatomically more complex, with elevated numbers among the elderly, diabetics, patients with renal failure and other illnesses. These patients most of the times need concomitant myocardial revascularization. For selected patients with bi-arterial lesions, the percutaneous coronary intervention can represent an alternative path of myocardial revascularization to surgery, capable of offering equivalent results in relation to the early and late life-span with less early morbidity. This way, around 40% to 60% of the percutaneous coronary intervention procedures are performed as bi-arterial treatment. These lesions can be coronary, vascular and renal.

These, additional, and/or other aspects and/or advantages of the present invention are: set forth in the detailed description which follows; possibly inferable from the detailed description; and/or learnable by practice of the present invention.

### BRIEF DESCRIPTION OF THE DRAWINGS

The present invention will be more readily understood from the detailed description of embodiments thereof made in conjunction with the accompanying drawings of which:

FIG. 1 shows the image of a patient in the bifurcation treatment —treatment of concomitant double artery

FIG. 2 shows the introduced constructive disposition in an indeflator device applied in coronary angioplasty interventions for bi-arterial treatment and/or Kissing-balloon technique, or double lesion, object of the present patent, highlighting all its configuration details.

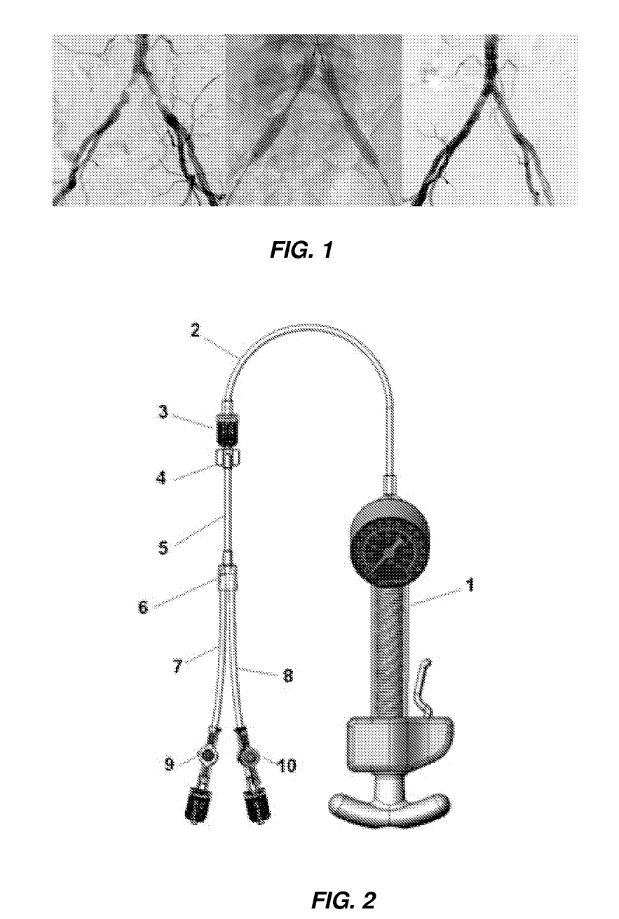

### DETAILED DESCRIPTION

Reference will now be made in detail to embodiments of the present invention, examples of which are illustrated in the accompanying drawings, wherein like reference numerals refer to the like elements throughout. The embodiments are described below to explain the present invention by referring to the figures.

In order to give veracity to the context set in the introductory depiction, an explanation will be presented on the state of the Kissing-balloon technique and the bifurcation lesion treatment, or bi-arterial.

The incremental development of this type of indeflator device is exactly to treat bifurcation lesions at the same time. Administrating liquids or fluids in the indicated pressure used during surgical procedures, to insufflate, control the pressure and also deflate the balloon's catheter, where this procedure is widely used in angiographic procedures, enabling the placement of a double endoprosthetic, also known as ‘stent’, for example. In a way that, the device can be used to inflate two balloons simultaneously as well as implant two endoprosthetics simultaneously.

On the other hand, the bi-arterial insuflator device presents its constructive concept comprised of the following main components: a Syringe; a syringe embolus; Manometer; inflation or deflation control system for the balloon type catheter; and a Double linking extension for the catheters connection.

Once the constructive concept of the insuflator device is recognized, it is pertinent to have a value analysis regarding its functional efficacy, where after several observations and notes one can conclude that, even though this device meets the prerequisites of “inflating, controlling the pressure and deflating a balloon type catheter, it also presents some flawed aspects, of constructive as well as operational nature.

### Proposal

With the exposition of the technique fundamental the disclaimer idealized a new introduced constructive disposition in insuflator device for the bi-arterial treatment, in which presents one entrance and two controlled exits, giving it a number of advantages with its use, for treatments and for hospital and medical procedures, due to its versatility, and greater comfort for the patient.

So, the following is a description of a non-restrictive preferential way to use the present device, object of this patent, which the configuration can vary to the required form for each model and desired application; describing one of the constructive possibilities that makes the described object concrete, and the way it works.

To complement the present description in a way that there is a better understanding of the present request's characteristics, attached is FIG. 2, where in an exemplified manner, although without limitations, represented the preferred way to use de bi-arterial indeflator device with a new control system of inflation or deflation of the simultaneous balloon type catheter, where: the introduced construction disposition in an indeflator device applied in coronary angioplasty interventions for the treatment of bi-arterial and the Kissing-balloon technique, or double lesion, object of the present patent, is essentially made of: an insuflator (1); a main tube (2); a male connector (3); a female connector (4); an extension tube (5); a “Y” connector (6); a bifurcation tube (7); another bifurcation tube (8); a terminal tap component (9); another terminal tap component (10); where the connecting component (3) can be a male or female connector; and the connecting component (4) can be a male or female connector; where the connection between the bifurcation tube (7) and the terminal tap component (9) can be a connection through connectors or glued; the connection between the bifurcation tube (8) and the terminal tap component (10) can be a connection through connectors or glued; and the connection between the main tube (2) of the insuflator (1) and the extension tube (5) can be glued or through the male or female connectors.

The introduced constructed disposition in an indeflator device applied in coronary angioplasty procedures for the treatment of bifurcation Kissing-balloon technique, or double lesion, object of the present patent, possesses a unique configuration with a bifurcation extension, in such a way that, the insuflator can work with two exits, controlled by two taps of 2 ways, identified by a colored buffer, of different colors, in each tap; The insuflator can work with two exits, being one controlled by a 3 way tap and the other with a rotating male connector; the insuflator can work with three exits, being two controlled by a 3 way tap, identified with different color covers in the taps, and the other with a rotating male connector; the insuflator can work with three exits, all three controlled by a 3 way tap each, identified with different color covers; and still the insuflator can work with three exits, all three controlled by a 2 way tap each, identified with different color covers on the taps.

This way, according to the description above, it can be noticed that the introduced constructive disposition in an indeflator device applied in coronary angioplasty interventions for the treatment of bi-arterial Kissing-balloon technique, object of the present patent, possess a bifurcation extension presented in 11 versions according to the matching of comprising elements, being those: the direct glue version, with three way taps; the male connection version, with a three way tap; the direct glue version, with a two way tap; the male connection version, with a two way tap; the direct glue version, with one two way tap and one three way tap; the male connection version, with one two way tap and one three way tap; the direct glue version, with only one two way tap; the male connection version, with only one two way tap; the male connection version, with only one three way tap; a direct glue version, with only one three way tap; and the male connection version, with two taps two way or three way. Giving great advantages in relation to the other insufflators used and found on the market today. Among these advantages we can name: meets the inflation requirements, pressure control and deflate a balloon type catheter, treat bifurcation lesions at the same time, and enable the placement of a double endoprosthetic, among others.

The introduced constructive device in an indeflator device applied in coronary angioplasty interventions for the bi-arterial treatment and Kissing-balloon technique, or double lesion, object of the present patent, that can also be presented with a trifurcation extension, with three ramifications with or without taps in the extremities, and can also be used for simple lesions, without bifurcation, using only one ramification and/or one tap, keeping the other one closed.

Thus, by the configuration and function characteristics, described above, we can clearly notice that the INTRODUCED CONSTRUCTIVE DISPOSITION IN AN INDEFLATOR DEVICE APPLIED IN CORONARY ANGIOPLASTY INTERVENTIONS FOR THE BI-ARTERIAL TREATMENT AND KISSING-BALLOON TECHNIQUE, it is a new device for the State of Technique which is protected by innovation conditions, unseen inventive activity and industrialization, that makes this invention worthy of the Patent Privilege of Utility Model.

Although selected embodiment(s) of the present invention have been shown and described, it is to be understood the present invention is not limited to the described embodiment(s). Instead, it is to be appreciated that changes may be made to these embodiment(s) without departing from the principles and spirit of the invention, the scope of which is defined by the claims and the equivalents thereof.

What is claimed is:

1. A method of constructive disposition introduced in a deflator device applied in coronary angioplasty interventions for bi-arterial treatment and a kissing balloon technique, characterized by an indeflator device to be used to insufflate two balloons simultaneously as well as implant two endoprosthetics simultaneously; which is essentially formed by: one insuflator (1); one main tube (2); a male connector (3); a female connector (4); an extension tube (5); a “Y” connector (6); a bifurcation tube (7); another bifurcation tube (8); a tap terminal component (9); another tap terminal component (10); where the connecting component (3) can be a male or female connector; and the connector component (4) can be a male or female connector; where the connection between the bifurcation tube (7) and the tap terminal tap component (9) can be by connection through connectors or glued; the connection between the bifurcation tube (8) and the tap terminal component (10) can be connected by connectors or glued; and the connection between the main tube (2) of the insuflator (1) and the extension tube (5) can be glued or through male or female connectors.

2. The method of claim 1, characterized by having a unique configuration with a bifurcation extension, in such way that, the insuflator can work with two ends, controlled by two taps with two ways, identified with a colored cover, of different colors, in each tap; the insuflator can work with both ends, one being controlled by a three way tap and the other with a rotating male connector; the insuflator can work with three ends, two being controlled by a three way tap, identified with different color covers, and the other with a rotating male connector; the insuflator can work with three ends, all three controlled by a 3 way tap each, identified with different color covers on the taps; and also the insuflator can work with three ends, all three controlled by a 2 way tap each, identified with different color covers on the taps.

3. The method of claim 1, characterized by presented with a trifurcation extension, with three ramifications with or without tap on their extremities.

4. The method of claim 1, characterized by the ability to be used in simple lesions, without bifurcation, using only one ramification and or one tap, keeping the other one closed.

5. The method of claim 2, characterized by the ability to be used in simple lesions, without bifurcation, using only one ramification and or one tap, keeping the other one closed.

6. An indeflator device usable to insufflate two balloons simultaneously as well as to implant two endoprosthetics simultaneously, the device comprising: one insuflator (1); one main tube (2); a male connector (3); a female connector (4); an extension tube (5); a “Y” connector (6); a bifurcation tube (7); another bifurcation tube (8); a tap terminal component (9); another tap terminal component (10); where the connecting component (3) can be a male or female connector; and the connector component (4) can be a male or female connector; wherein the connection between the bifurcation tube (7) and the tap terminal tap component (9) is by connection through connectors or adhesive, the connection between the bifurcation tube (8) and the tap terminal component (10) is by connection through connectors or adhesive, and the connection between the main tube (2) of the insuflator (1) and the extension tube (5) is glued or through male or female connectors.
